(19) United States
(12) Patent Application Publication
(10) Pub. No.: US 2005/0011762 A1
(43) Pub. Date: Jan. 20, 2005

(54) Bottom access electrophoresis tray and method of use

(76) Inventor: Richard B. Provonchee, Cushing, ME (US)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/869,555

(22) Filed: Jun. 15, 2004

Related U.S. Application Data
(60) Provisional application No. 60/483,007, filed on Jun. 26, 2003.

(51) Int. Cl. G01L 1/20; C07K 1/26

(52) U.S. Cl. 204/451; 204/600

(57) ABSTRACT

An electrophoresis gel tray having bottom electrical field access and a method of running a gel are disclosed. The tray includes a gel base having an electrical field ingress port and an electrical field egress port disposed proximate opposite ends of the gel base. In use, the tray is placed on a support in an electrophoresis running tank. The running tank is filled with a buffer solution to a level that is at least even with the gel base. With the tray and buffer solution in place, electrical current is applied to the buffer solution. The field enters the tray through the electrical field ingress port, flows through the gel and exits the gel through the electrical field egress port, thereby effecting electrophoretic separation of one or more samples placed in the wells of the gel.

### RELATED APPLICATION

This application is related to and claims priority from U.S. Provisional Patent Application No. 60/483,007, filed Jun. 26, 2003, which is incorporated herein by reference in its entirety.

### FIELD OF THE INVENTION

The present invention relates to the field of electrophoresis and, more particularly, to a tray for an electrophoresis gel and method of running the gel.

### BACKGROUND OF THE INVENTION

Gel electrophoresis is a process that has long been used for clinical diagnosis and laboratory research. It is based upon the principle that electrically charged biological macromolecules will migrate through a solvent medium when subjected to an electrical field. Since macromolecules vary in molecular weight and charge, it is possible to use an electrophoresis process to separate the macromolecules and distinguish between them based on their respective rates of movement through the medium. Electrophoresis can also be used for other types of macromolecular analysis, such as detecting amino acid changes.

In a common form of gel electrophoresis, the gel solution is cast and solidifies into a thin planar slab gel. The gel is placed in a buffer solution within an electrophoresis gel chamber, also known as a running tank. The samples to be tested are then placed within cavities or wells formed in the electrophoresis gel. A current is applied to the buffer solution causing the biological macromolecules to migrate through the gel.

Originally, the laboratories conducting the testing mixed the gel solution and cast their own gel slabs on-site. It soon became apparent, however, particularly as electrophoresis testing of DNA became common, that it is more convenient and more precise to use precast gel slabs made to uniform composition, size and configuration standards. The most common precast gel slab has a thin planar rectangular shape and includes a series of spaced wells which receive the biological samples being investigated. Conventional gel slabs are inherently flimsy and subject to tearing and deformation if not handled carefully. A particularly sensitive area in the gel is the thin walls separating the sample wells. While any deformation or tearing of the gel slab creates some risk of producing inaccurate results, a breach between wells allowing commingling of adjacent biological samples could generate erroneous results.

Thus, precast gels have been supplied in trays to protect them from mechanical damage. While the trays provide a suitable mechanism for protecting the electrophoresis gel from damage, the trays typically do not include a convenient mechanism for holding the gel submerged under the buffer solution within the gel chamber. Since the gel is nearly the same density as the buffer solution, small movements of the chamber can easily cause the gel to shift. Also, any slight movement of the overlaying buffer solution can cause the gel to shift. The motion of the buffer solution can be caused by thermal gradients produced in the buffer by the electric current, or by bubble generation in the buffer. Shifting of the gel in the running tank is sometimes referred to as drifting or floating. One device recently developed to hold down a gel slab during an electrophoresis process is an anchor disclosed in U.S. Pat. No. 6,106,686, which is incorporated herein by reference. The anchor includes a plurality of supporting members (legs) which extend downward from a frame. The supporting members are positioned so as to rest on the top of the gel during the electrophoresis process. While this anchoring device is a convenient and easy solution to the floating problem, there are situations where it may not be desirable to use a distinct anchor device to hold the gel in place.

Another problem associated with conventional precast gel trays is that they increase the chances of developing artifacts in the gel. In particular, the use of a tray during an electrophoretic run can produce an effect known as “hourglassing”, as well as result in the appearance of tilted bands in the gel. Hourglassing is a problem caused by the presence of the tray itself during a run. In general, trays are formed from electrically inert materials. The tray disrupts the flow of electrical current from the buffer into and out of the gel, thereby producing a non-uniform electrical field. The deleterious effects of hourglassing and tilted bands are more fully shown and described in U.S. Pat. No. 6,328,870, which is incorporated herein by reference.

One attempt to alleviate the hourglassing problem is to form a tray with open ends through which the electrical field can flow unimpeded into and out of the gel. However, such a configuration tends to make a tray structurally unsound. Thus, trays with open ends must be relatively thick to ensure adequate stability and structural integrity. Unfortunately, these thick plastic trays must be formed by injection molding; and are, therefore, expensive to produce. Moreover, the open-ended trays do not solve the problem of drifting since the trays must be submerged in the buffer solution during a run to achieve electrical flow through the open sides.

A need still exists for a tray for an electrophoresis gel that can be inexpensively produced and that alleviates both the hourglassing and drifting problems.

### SUMMARY OF THE INVENTION

The present invention relates to an electrophoresis gel tray having bottom electrical field access. The invention further relates to a method of running an electrophoresis gel by introducing an electrical field through the bottom of the tray.

The bottom access tray according to the present invention includes a gel base having a bottom surface and a gel engaging top surface. The tray also preferably includes a substantially uninterrupted wall extending upwardly from the periphery of the gel engaging surface. To provide for the introduction of the electrical field, an electrical field ingress port and an electrical field egress port are disposed proximate opposite ends of the gel base flush with the bottom and top surfaces.

According to the method of the present invention, a tray is placed in an electrophoresis running tank on a support such that the access ports are not obstructed by the support. The running tank is filled with a buffer solution to a level that is at least even with the gel base. The buffer solution may be filled to a higher level, so that the top of the tray is even with the surface of the buffer solution surface or so that the tray is submerged. However, it is not necessary to completely submerge the tray. With the tray and buffer solution in place, an electrical field can be applied to the buffer solution. The electrical field flows upwardly into the gel through the ingress port, horizontally through the gel and downwardly out of the gel through the egress port.

### BRIEF DESCRIPTION OF THE DRAWINGS

For the purpose of illustrating the invention, there is shown in the drawings a form which is presently preferred; it being understood, that this invention is not limited to the precise arrangements and instrumentalities shown.

FIG. 1 is an isometric view of a precast gel in a gel tray according to the present invention.

FIG. 2 is a top plan view of the precast gel and gel tray of FIG. 1.

FIG. 3 is a cross-sectional view of the gel and tray as seen through line 3-3 of FIG. 2.

FIG. 4 is a cross-sectional view of the gel and tray as seen in FIG. 3, the tray having been placed on a support in a running tank.

FIG. 5 is the cross-sectional view of the gel and tray of FIG. 4, showing the propagation of an electrical field through the tray.

FIG. 6 shows a running tank equipped with a cross bar.

### DETAILED DESCRIPTION OF THE DRAWINGS

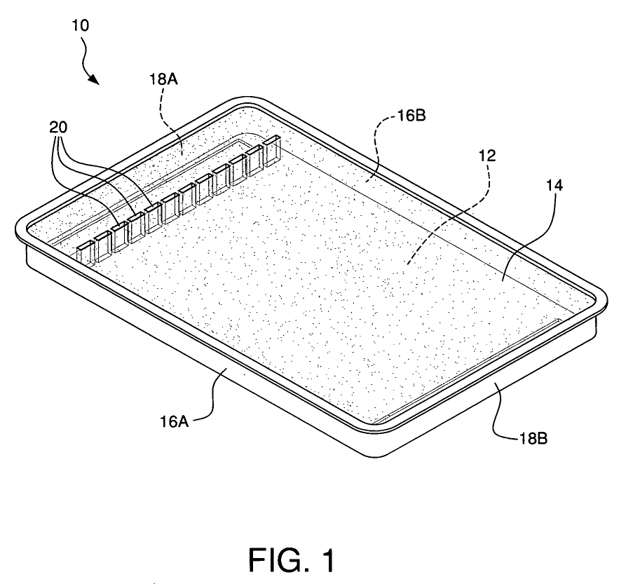

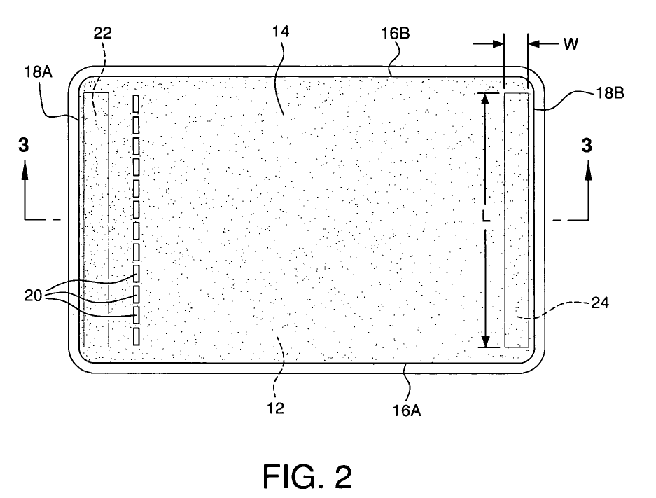

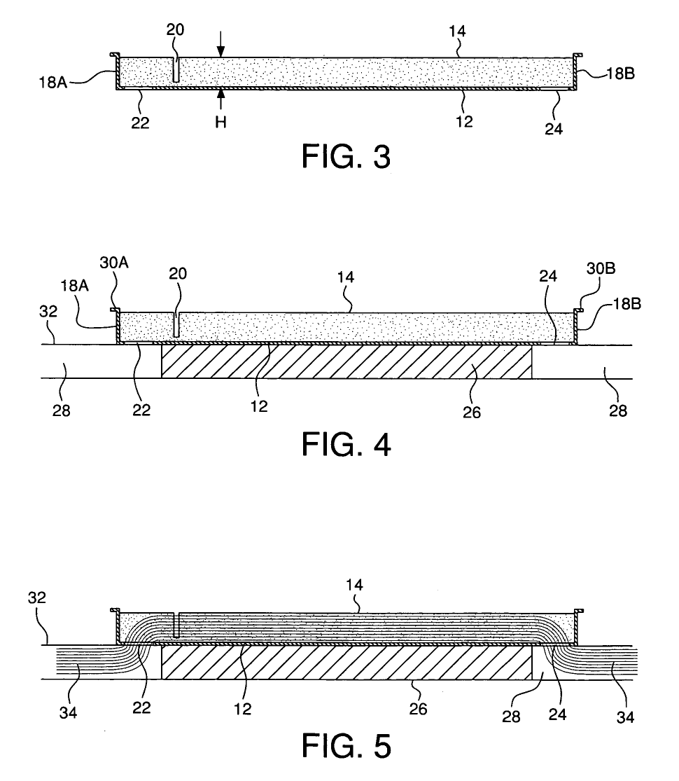

Referring now to the drawings, wherein like reference numerals illustrate like elements throughout the several views, FIGS. 1-5 show a preferred embodiment of an electrophoresis gel tray with bottom electrical field access. As shown in FIGS. 1 and 2, the tray 10 includes a bottom gel base 12 with a top surface for supporting an electrophoresis gel 14. The tray has two major sides 16A and 16B extending upwardly from opposite sides of the gel base 12. The tray further includes two minor sides 18A and 18B extending upwardly from opposite sides of the gel base 12 and connecting the major sides 16A and 16B. The major sides 16 and minor sides 18 collectively form a substantially uninterrupted wall extending upwardly from the periphery of the gel engaging surface of the gel base 12. The electrophoresis gel 14 is preferably of the precast type and includes sample wells 20. (The terms “major” and “minor” are simply used here for convenience and are not intended to suggest any size limitation on the present invention.)

The gel base 12 includes electrical field access ports proximate opposite ends of the gel base. An electrical field ingress port 22 is provided proximate minor side 18A between the sample wells 20 and the minor side 18A. An electrical field egress port 24 is provided in the opposite end of the gel base 12 proximate minor side 18B. As shown in the drawings, the ingress port 22 and egress port 24 can be in the form of rectangular slots extending most of the width of the gel base 12. However, each port can, instead, be formed in a variety of different shapes, a row of circular or polygonal apertures, or two or more elongated rectangular openings being just a few examples. A row of apertures can provide the tray 10 with greater structural integrity than a tray with long rectangular ports. However, when forming the tray with such apertures, space between the apertures should be minimized so that electrical current can flow through the gel substantially uniformly across the width of the tray.

On the other hand, it may be advantageous to intentionally vary the cross section of the ingress and egress ports across the width of the tray. For example, it may be desirable to decrease the width of the ports near their mid-point. During a run, the center portion of a gel tends to become hotter than the outer portions due to disproportionate rates of heat dissipation among these sections. (The outer portions of a gel can dissipate heat at a faster rate than the center of the gel.) Because the center portion tends to become hotter, separation of the molecules under analysis can happen faster in the center of the gel. Thus, samples loaded near the center of the gel can appear to migrate faster than those near the outer portions of the gel. By reducing the width of the ingress and egress ports at the center, less current will flow through the center of gel, thereby both reducing the heat gradient across the width of the gel and reducing the rate at which separation occurs at the center. Alternatively, if each port is formed from a row of apertures, the apertures can be made smaller near the center of the row or can be separated near the center of the row with more space than those at the ends of the row. In the case of rectangular ports, like those shown in the drawings, the ports can be modified by interrupting them near their mid-points with solid portions of the gel base 12 to achieve a similar affect. Using any of these configurations, a more uniform separation of molecules across the width of the gel may be achieved during an electrophoresis run. That is, the samples being run in the center of the gel can be separated at a rate very close to those being run in the outer portions.

In addition to shape, the size of the ingress 22 and egress 24 ports is also important. The ingress 22 and egress 24 ports should be large enough to allow substantially uniform electrical field propagation through the gel, yet not so large as to interfere with the structural integrity of the tray. It is presently contemplated that the ingress 22 and egress 24 ports can be rectangles having a length, L, that is close to the width of the tray 10. The width, W, of the ports can be close to the height, H, of the gel 14 disposed within the tray. Testing has established that selecting a width for the ports that is close to the height of the gel 14 allows for the desired uniform propagation of the electrical field through the gel. It is also possible to decrease the width of the ports in order to increase the structural integrity of the tray. Decreasing the width of the ports, however, locally restricts the flow of current to a smaller cross-sectional area. Therefore, it may be necessary to increase the electric potential across the system to achieve the same rate of separation. If the width of the ports is decreased too much, excessive heat may be generated. At present, it is believed that the width of the ports can be reduced to about one half of the height of the gel 14 without generating unacceptable amounts of heat. The width of the ports can also be greater than the height of the gel. However, increasing the width of the ports may affect the structural integrity of the tray. Thus, it is preferred that the width of the ports be no more than about twice or three times the height of the gel.

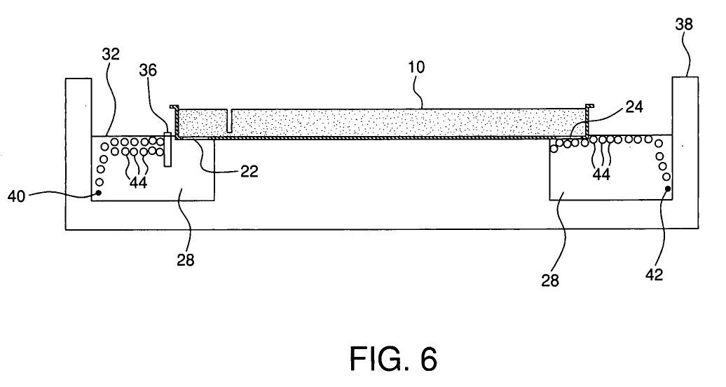

In another embodiment (not shown), the gel base 12 can be formed with sloped portions between each of the ingress and egress ports and the their respective proximate sides. For example, the gel base 12 may be angled upwardly toward the minor side 18A between ingress port 22 and the minor side 18A. Similarly, the gel base 12 may be angled upwardly toward the minor side 18B between egress port 24 and the minor side 18B. The sloped portions of the gel base 12 will assist air in escaping from under the tray 10 when it is placed in the running tank. Thus, the sloped portions can ensure that no air bubbles interfere with the interface between the buffer solution in the tank and the gel 14 at each of the ingress 22 and egress 24 ports. To further protect against air bubbles interfering with the interface between the buffer solution and the gel, the running tank can also be configured to accept two cross bars, which will be described below with reference to FIG. 6.

The substantially uninterrupted wall extending from the periphery of the gel base 12 provides the tray with good structural integrity. Thus, the tray 10 can be formed with relatively thin plastic using an economical thermoforming process. In such a process, a sheet of thermoplastic is heated to its processing temperature and drawn onto a shaped mold by a vacuum, for example. The heated sheet takes on the shape of the mold and is cooled in that shape. Preferred materials are those which are chemically inert and are not electrically conductive. Of course, the selected material should also withstand temperatures associated with the electrophoresis process and be rigid enough to adequately support the gel 14. Suitable materials can include polystyrene, high density polyethylene, low density polyethylene, linear low density polyethylene, polyethylene naphthalate, polyvinyl chloride, polyvinylidene chloride, polycarbonate, polymethylmethacrylate, polyvinylacetate, ethylene vinylacetate, polypropylene, some polyesters, such as polyethylene terephtalate (PET) and glycol-modified PET, cellulose acetates, polyamides, and copolymers thereof.

The tray 10 of the present invention is preferably provided with a precast gel 14 as shown in the drawings. The gel may be an agarose gel for separation of, for example, DNA with about 100 or more base pairs, or a polyacrylamide gel for separation of smaller nucleic acids or biological proteins. However, it is also possible to provide the tray 10 without the gel, thereby allowing the end user to cast the gel on-site. In order to form the gel, a comb should be supported extending most of the depth of the tray in order to form the sample wells 20 while the gel is poured and solidified. The access ports can be covered with tape or other removable cover while the gel is poured and cooled. This method will form gel within the ingress 22 and egress 24 ports so that the solidified gel is flush with the bottom surface of the gel base 12 once the tape or cover is removed. Alternatively, port stoppers can be employed which completely seal the ingress and egress ports 22, 24 while the gel is poured and solidified. If port stoppers are used to seal the ports, the gel will be flush with the top surface of the gel base 12. In the latter case, care must be exercised to ensure that no air bubbles form in the ports when the tray is placed in the running tank.

In use, the tray 10 is placed in a running tank. As shown in FIG. 4, the running tank is provided with a support 26, on which the tray 10 can be placed. The support 26 can be a simple platform or any other type known to those skilled in the art. However, it is important that the support does not obstruct the ingress 22 and egress 24 ports. It has already been noted that electrophoresis can create heat within the system. In fact, excess heat has been known to denature the samples being analyzed in the process. Thus, it is preferable that support 26 be formed from a thermally conductive material and act as a heat sink to help draw heat away from the gel base 12 and gel 14 during the run. If desired, the support 26 can, in turn, be thermally coupled with a cooling device, for example, a heat sink or thermoelectric device, to further enhance heat draw.

In an alternative embodiment, the support 26 can be hollow (not shown) so as to provide an isolated or semi-isolated buffer reservoir to help evenly dissipate heat. Such an alternative support can be of approximately the same dimension as support 26 shown in FIG. 4, but in the form of a box that is open at the top. It is preferred that the buffer within the alternative support is substantially electrically isolated so that little or no heat is produced within the support during the run. However, minimal fluid communication may be desirable between the buffer within the alternative support and buffer in the remainder of the running tank, described immediately below, in order to self-regulate the level of buffer within the support, while allowing only negligible electrical flow therethrough.

The buffer solution in the running tank is labeled as element 28 in the drawings. The level 32 of the buffer 28 need only be provided to the gel base 12 where gel 14 fills the ingress 22 and egress 24 ports (e.g., when the gel is poured into the tray using the above-described taping method). However, the buffer solution 28 should be filled slightly higher and care should be taken to avoid air bubbles if the gel does not occupy the ingress 22 and egress 24 ports. It has also been determined that the current tray can work with the buffer level somewhat below the level of the ports. If properly positioned, surface tension will draw the buffer up to the ports. In addition, other than the use of extra buffer, there is no particular harm in filling the buffer solution to a level between the gel base 12 and the top of the wall lips of the tray, indicated in FIG. 4 by the reference numerals 30A and 30B. In addition, if desired, the tray 10 of the present invention can be used in a running tank filled with buffer beyond the level of the wall lips 30A and 30B. In the latter circumstance, the tray 10 is used in a manner very similar to conventional “submarine” electrophoresis. Even when so used, it is believed that the tray 10 is advantageous over prior known trays because the bottom electrical field access provided by ingress port 22 and egress port 24 helps provide a more uniform electrical field than does a conventional electrophoresis tray.

However, it is preferred that the running tank be filled with buffer solution 28 to a point under the lips 30A and 30B because further filling is believed to be unnecessary. Also, buffer solution is generally expensive and can be environmentally unfriendly. Thus, it is recommended that the solution be filled only to, or slightly above, the level of the gel base 12.

By filling the buffer solution 28 in the running tank to the recommended level, a user may take advantage of several additional advantages associated with the present invention. One such advantage is that samples can be loaded into the sample wells 20 prior to placing the gel into the running tank. Thus, the sample wells 20 can be loaded in a convenient place in the most suitable manner. Loading the samples prior to placing the tray in the running tank avoids the need to load the samples in the tank, which can be awkward. In conventional submarine electrophoresis, it is impractical to load the sample wells prior to placing the gel into the running tank because the act of submerging the gel into the buffer can displace sample from one well to another, thereby cross contaminating the samples in the various wells. Instead, in a conventional submarine system, one must first submerse the gel into the buffer before loading the sample into the wells. In general, the samples are mixed with dye or buffer in order to make relatively dense samples solutions that will not diffuse out of the top of the sample wells.

Once the gel is ready to be run, a cathode (shown in FIG. 6 as element 40) is placed in the buffer solution on the side of the support that is in contact with the electrical field ingress port 22 (i.e., on the left side of FIG. 5). An anode (shown in FIG. 6 as element 42) is placed on the opposite side of the support 26 in the buffer solution in contact with electrical field egress port 24 (i.e., on the right side of FIG. 5). When the system is energized, current flows from the cathode to the anode, which generates an electrical field. In FIG. 5, the path of the current and the electrical field are shown by flow lines 34. The field flows through the buffer 18 upwardly through the ingress port 22 and into the gel 14. The field then flows horizontally through the gel 14 to the egress port 24. There, the field flows downwardly through the egress port 24 into the buffer solution.

Because the gel can be run with the buffer filled only to the level 32, less current is needed to run the gel than would be required if a conventional tray were used. In a conventional submarine system, electrical current flows across the top and around, as well as through the gel. On the other hand, if the buffer is filled only to a level below lips 30, such as level 32, a larger proportion of the current passes through the gel, rather than flowing above or around it. Thus, less current can be used at a given voltage to achieve the same run time. Because less current is required, less heat will be generated within the system.

Yet another advantage of the present invention is that the tray 10 is not susceptible to the problem of drifting or floating. Like in a conventional tray, the gel used in the tray 10 may have about the same density as the buffer solution surrounding the tray. Also, since the tray 10 can be formed by an economical thermoforming process, the tray itself may not add significant weight to the system. Thus, one might expect to need an anchoring device to keep the tray in place. However, because the buffer solution 28 in the running tank can be filled to a level below the lips 30A and 30B, such as level 32, the unsubmerged portions of the tray 10 and gel 14 represent weight that is unbalanced by buffer. Thus, the unsubmerged portions provide adequate force to prevent the tray 10 from drifting or floating without the need for any separate anchoring device.

As mentioned above, FIG. 6 shows a running tank 38 configured to accept two cross bars 36 that sit partially in the buffer 28 just beyond the ends of the tray 10 in order to further protect against air bubbles interfering with the interface between the buffer solution and the gel. In FIG. 6, the running tank 38 is equipped with only one such cross bar 36, disposed between the electrical field ingress port 22 and the cathode 40. FIG. 6 does not show a second cross bar 36 in order to more clearly demonstrate the advantages and function of the cross bars. The cross bars 36 act to block gas bubbles 44, which are generated at the cathode 40 and anode 42 during the run, from reaching the ingress 22 and egress 24 ports. Gas bubbles 44 generated at the electrodes tend to rise to the surface of the buffer and can travel horizontally along the surface 32 of the buffer. As shown on the right hand side of FIG. 6 (near the anode 42), without a cross bar 36, the gas bubbles 44 are free to travel horizontally until they contact the edge of the tray 10. If the buffer level is right at or near the bottom of the tray, these bubbles 44 can collect at the ingress 22 and egress 24 ports (only the egress port 24 in the drawing) and disrupt current flow.

However, as demonstrated on the left side of FIG. 6, the inclusion of a cross bar 36, partially submerged in the buffer between the cathode 40 and the ingress port 22, can block the horizontal travel of the gas bubbles 44, thereby keeping the bubbles clear of the ingress port 22. A second cross bar (not shown) similarly disposed at the surface 32 of the buffer between the anode 42 and the egress port 24 can reduce or eliminate gas bubbles at the egress port 24.

Cross bars of an approximately 6 mm square cross section have been found to be suitable. However, the size and cross sectional shape of the cross bar can vary. It can be rod shaped, square, rectangular, etc. The lower edge of the bar should be low enough to catch any bubbles 44 but not so close to the bottom of the chamber as to restrict current flow to the gel. The upper edge of the bar should be at least at a level of about 1 mm above the bottom edge of the tray. As the buffer level is raised to be closer to the level of the wall lips 30 of the tray, the cross bars 36 can also be raised. Another possibility is to utilize floating cross bars that ride in vertical grooves or between two ribs on each side of the tank wall. A crossbar that is properly weighted to exhibit appropriate buoyancy in the buffer can prevent horizontal migration of bubbles 44 regardless of the buffer level in the running tank.

A variety of modifications to the embodiments described will be apparent to those skilled in the art from the disclosure provided herein. Thus, the present invention may be embodied in other specific forms without departing from the spirit or essential attributes thereof and, accordingly, reference should be made to the appended claims, rather than to the foregoing specification, as indicating the scope of the invention.

1. A method of running a horizontal electrophoresis gel, the method comprising the steps of:
providing an electrophoresis gel in a tray having a height, a bottom electrical field ingress port and a bottom electrical field egress port;
loading one or more samples into sample wells formed in the gel;
filling a running tank with a buffer solution to a level between the top of a gel support within the tank and the height of the tray above the support;
placing the gel on the support in the running tank; and
applying an electrical field through the running tank, the electrical field flowing upwardly through the ingress port, horizontally through the gel and downwardly through the egress port.

2. The method of claim 1 wherein the step of placing the gel is performed after the loading step.

3. The method of claim 1 wherein the providing step comprises the steps of thermoforming the tray and casting the gel in the tray.

4. The method of claim 3 further comprising the steps of covering the ingress port and egress port with tape prior to casting the gel.

5. An electrophoresis tray comprising:
a gel base having a bottom surface and a gel engaging top surface;
a substantially uninterrupted wall extending upwardly from the periphery of the gel engaging surface; and
an electrical field ingress port and an electrical field egress port disposed proximate opposite ends of the gel base, the ingress and egress ports being flush with the bottom surface of the gel base.

6. The electrophoresis tray of claim 5 further comprising a precast electrophoresis gel having a height, the ingress and egress ports each having a width that is within the range of from about half of the height of the gel to about twice the height of the gel.

7. The electrophoresis tray of claim 5 wherein the gel base and the wall are integrally formed in a thermoforming process.

8. The electrophoresis tray of claim 7 wherein the ingress and egress ports are formed integrally in the gel base during the thermoforming process.

9. The electrophoresis tray of claim 5 wherein the ingress and egress ports each comprise a substantially rectangular slot in the gel base, each slot extending for most of the width of the gel base.

10. The electrophoresis tray of claim 5 wherein the ingress and egress ports each comprise a plurality of slots in the gel base.

11. The electrophoresis tray of claim 5 wherein the ingress and egress ports each comprise a row of apertures in the gel base.

12. A method of running an electrophoresis gel, the method comprising the steps of:
providing an electrophoresis tray having a gel base with an electrical field ingress port and an electrical field egress port disposed flush in the gel base proximate opposite ends of the gel base, a substantially uninterrupted wall extending upwardly from the periphery of the gel base, and a gel resting on the gel base within the wall;
running an electrical current upwardly into the gel through the ingress port, horizontally through the gel and downwardly out of the gel through the egress port.

13. The method of claim 12 further comprising the steps of placing the gel in a running tank and filling the running tank with buffer solution to a level at least as high as the gel base, but not higher than the top of the gel.

14. The method of claim 13 further comprising the step of loading samples into sample wells in the gel before placing the gel in the running tank.

15. The method of claim 12 wherein the providing step comprises the steps of thermoforming the electrophoresis tray, obstructing the ingress and egress ports and pouring the gel.

16. A method of running an electrophoresis gel comprising the steps of:
providing an electrophoresis tray having a gel supported on an gel base with an electrical field ingress port and an electrical field egress port;
positioning the tray in a running tank having electrodes near opposite ends and being partially filled with buffer such that the electrical field ingress port and electrical field egress port are near the surface of buffer;
placing a cross bar in the running tank at the surface of the buffer between an electrode and the electrical field ingress port or electrical field egress port;
running an electrical current upwardly into the gel through the ingress port, horizontally through the gel and downwardly out of the gel through the egress port, the electrical field flowing underneath the crossbar.
